(19) United States
(12) Patent Application Publication
(10) Pub. No.: US 2010/0255118 A1
(43) Pub. Date: Oct. 7, 2010

(54) ORAL ZINC MEDICANTS USEFUL FOR SAFELY LOWERING FREE COPPER ABSORPTION AND FREE COPPER LEVELS

(76) Inventor: Steve H. Kanzer, Ann Arbor, MI (US)

Correspondence Address: GARVEY SMITH NEHRBASS & NORTH, LLC, LAKEWAY 3, SUITE 3290, 3838 NORTH CAUSEWAY BLVD., METAIRIE, LA, 70002, US

(21) Appl. No.: 12/530,994

(22) PCT Filed: Mar. 12, 2008
(86) PCT No.: PCT/US08/56743

§ 371 Date: Jun. 2, 2010

Related U.S. Application Data
(60) Provisional application No. 60/894,388, filed on Mar. 12, 2007.

(51) Int. Cl. A61K 33/34; A61K 33/30; A61P 3/02; A61K 9/22; A23L 1/304

(52) U.S. Cl. 424/630; 424/643; 426/89

(57) ABSTRACT

A preparation preferably containing zinc and sustained release copper. Additional nutrients can be included in the preparation.

### CROSS-REFERENCE TO RELATED APPLICATIONS

Priority of U.S. Provisional Patent Application Ser. No. 60/894,388, filed 12 Mar. 2007, incorporated herein by reference, is hereby claimed.

Also incorporated herein by reference are U.S. patent application Ser. No. 11/621,962, filed 10 Jan. 2007, and published on 6 Sep. 2007 as patent publication no. US2007/0207191 A1, and International Patent Application No. PCT/US2007/060345, filed 10 Jan. 2007, and published on 26 Jul. 2007as patent publication no. WO 2007/084818 A2.

This is not a continuation or continuation-in-part of any patent application.

### STATEMENT REGARDING FEDERALLY SPONSORED RESEARCH OR DEVELOPMENT

Not applicable

### REFERENCE TO A “MICROFICHE APPENDIX”

Not applicable

### BACKGROUND OF THE INVENTION

1. Field of the Invention

The present invention is directed to multi-vitamin and mineral supplements. More particularly, the invention is related to multi-vitamin and mineral supplements for improving health by insuring adequate intake of copper, iron and/or zinc. The invention has particular applicability to treating or preventing age-related conditions such as macular degeneration and cognitive decline and will be described in connection with such utility, although other utilities are contemplated.

2. General Background of the Invention

Vitamin and mineral preparations commonly are administered to treat specific medical conditions and/or as a general nutritional supplement. Micronutrients are elements or compounds that are present in foods in small or trace amounts and include vitamins, minerals and other elements and compounds found in foods in which a Recommended Daily Allowance (RDA) has not as yet been determined. Micronutrients also include carbohydrates, fats, and proteins which supply nutrients and calories. Some elements such as calcium, sodium, potassium, chloride and phosphorus are consumed in relatively large amounts, while elements such as copper, iron, and zinc are consumed in relatively small amounts. Inasmuch as humans (and animals) do does not synthesize many compounds which are essential to health, these specific vitamins, minerals and elements can be obtained only from two sources: food and supplements. The primary source of all nutrients is food. However, a majority of humans (and animals) do not intake the RDA of the foods containing essential compounds and elements. Thus, vitamin and mineral supplementation has become a recognized method of meeting accepted medical and health standards.

Recent studies have illustrated the important physiological roles played by vitamins and minerals and established a correlation between deficiencies or excesses of this nutrients and the etilogies of certain disease states in humans.

Copper and iron are essential trace elements but are also the primary oxidants to people. Since copper and iron are necessary, their intake is recommended. Copper and iron enter the blood with the flux of water resulting in elevated free copper and iron in blood. In the elderly, copper and iron also may enter the brain due to the leaky blood brain barrier. It is a function of the liver to process excess copper and iron in the blood (and brain) and bring these toxins down. However, processing is not instantaneous, and elderly people who generally have impaired liver function, and younger people with impaired liver function, the levels of copper and iron oxidants often stay elevated much longer and may cross the blood/brain barrier.

Moreover, despite its essentiality, however, copper also is an extremely reactive oxidative species that has the potential to be very toxic to cells, proteins, and organ systems such as the liver, brain and vasculature. In order to deliver and utilize copper in the body on demand wherever it is needed, mammalian systems have developed an elaborate regulatory network of highly specific, homeostatic, copper-binding cuproproteins that serve to properly scavenge, store, transport, chaperone and excrete copper while minimizing the potential for copper to inadvertently oxidize or reduce proteins and lipids. Many different cuproproteins have been identified and their functions have been elucidated over the years. Examples of such cuproproteins include, but are not limited to, matrix metalloprotein, ceruloplasmin, copper/zinc superoxide dismutase, amyloid precursor protein, apolipoprotein E, tau, homocysteine, albumin and chaperone for copper zinc superoxide dismutase, to name a few.

One of the most problematic and potentially toxic sources of copper for humans is the abundance of toxic copper ions that exists in drinking water systems. Unlike the copper found in food, which is bound to proteins and the absorption of which is relatively easily regulated by the intestines and the slow dissolution from food during digestion, copper in drinking water occurs in the form of cupric ion (Cu⁺²) in either an unbound form or in a form complexed only loosely with organic ligands. Copper ions are generally more bioavailable in water than they are in food; there may be components in food that can influence the metabolism, absorption and mobilization of copper in human diets.

### Absorption of Copper by the Human Body

In humans, dietary copper is absorbed from the stomach and small intestine. In one study, about 65 percent of an oral dose of Cu⁶⁴ as copper acetate was absorbed from the gastrointestinal tract of humans (range 15-97 percent) (Weber et al., 1969; Strickland et al., 1972). Absorption efficiency appeared to be inversely correlated with copper level in the diet (Turnland et al., 1989, 1998). Orally administered Cu⁶⁴ rapidly appears in the plasma (Beam and Kunkel, 1955).

Dorner et al. (1989) found that full-term, breast-fed human infants, with a copper intake of 114 μg/kg-day, retained 88 μg/kg-day of copper, representing an absorption value of ˜77 percent. Copper retention decreased with age. At two weeks of age, 130 μg/kg-day was retained, and at age 16 weeks, 64 μg/kg-day was retained. In comparison, mean relative retention in infants fed copper-fortified formula was 52 percent. Copper absorption in infant rhesus monkeys using Cu⁶⁷ ranged from 50-70 percent, similar to the values found for full-term human infants (Lonnerdal et al., 1996). Studies in rats show that copper absorption is very high during the neonatal period, but that it decreases by the weaning period (Lonnerdal et al., 1985). Using perfused rat intestines, Varada et al. (1993) found that copper absorption was linear and nonsaturable in infant and weanling rats, and copper absorption was saturable in adolescent rats. Suckling rats had considerably higher tissue copper concentrations than weanling or adolescent rats. Citrate, a dietary food-ligand found in human and cow milk, has been shown to have a positive effect on copper absorption in animal models (Shah, 1981).

Olivares et al. (2002) administered an oral supplementation of 80 μg Cu (as copper sulfate solution)/kg daily for 15 days to a group of Chilean infants aged 1-3 months (n=20); one half of the group (n=19) received no supplementation. At the end of the trial, copper absorption was measured by using orally administered Cu⁶⁵ as a tracer and fecal monitoring of recovered Cu⁶³. No major difference in the percentage of copper absorbed was observed between the two groups. Mean (+SD) copper absorption at one month of age was 83.6+5.8 percent and 74.8+15.2 percent for the unsupplemented and supplemented infants, respectively. The authors concluded that the experimental design of the study was inadequate because copper intakes were too low to “trigger homeostatic adaptation of intestinal absorption.”

Copper absorption in the gastrointestinal tract has been studied in rats and hamsters. Absorption takes place from the stomach and duodenum in rats (Van Campen and Mitchell, 1965) and from the lower small intestine in hamsters (Crampton et al., 1965). Copper absorbed from the gastrointestinal tract may be bound to amino acids or may be in the form of ionic copper. Copper becomes bound to metallothionein in the intestine and may be either absorbed or sloughed off back into the intestinal lumen.

The existence of a protein source (plant or animal protein), amino acids, carbohydrates and/or ascorbic acid can affect copper availability (Gibson, 1994; Lonnerdal, 1996). Competition with zinc and cadmium affects copper absorption from both diet and drinking water (Davies and Campbell, 1977; Hall et al., 1979). Ascorbic acid may alter the metallothionein binding site. High dietary ascorbic acid has been shown to interfere with absorption of copper in guinea pigs (Smith and Bidlack, 1980), but this does not appear to be a factor at the usual ascorbic acid doses in humans (Jacob et al., 1987). Phytates and fiber interfere with copper absorption by forming complexes with copper (Gibson, 1994). The amount of copper stored in humans, which is mainly in the liver, does not appear to affect copper absorption (Strickland et al., 1972). There do not appear to be any available studies of copper absorption in humans by inhalation.

Batsura (1969) observed copper oxide in alveolar capillaries after rats were exposed to welding dust from a pure copper wire. No studies of the rate or extent of absorption of copper through intact human skin were found, but as copper can cause contact dermatitis, some absorption must occur (ATSDR, 1990). Pirot et al. (1996) studied the absorption of copper and zinc through human skin in vitro. Skin absorption is not likely to contribute significantly to total copper absorption.

### Distribution of Copper in the Human Body

Copper is transported in the plasma and is bound to ceruloplasmin, albumin or amino acids (Cousins, 1985). Ceruloplasmin is a cysteine-rich glycoprotein with many free sulfhydryl groups that serve as binding points for metals. Ceruloplasmin can bind copper or zinc, but has a stronger affinity for copper (Cousins, 1985). Ceruloplasmin is synthesized on membrane-bound polyribosomes of liver parenchymal cells and secreted into the plasma. Copper that enters the portal circulation from the intestine is transported directly to the liver. Copper released from the liver is transported in the bloodstream to other organs, including the kidney and brain. The synthesis of ceruloplasmin is controlled by interleukin-I via glucagon or glucocorticoid (Cousins, 1985). Circulating copper levels are elevated in pregnant women because hormonal changes associated with pregnancy stimulate ceruloplasmin synthesis (Solomons, 1985). Ceruloplasmin levels may be useful as an indicator of copper status (Mendez et al., 2004).

Recently, several copper transporters involved in copper uptake and transport by cells have been identified (Bauerly et al., 2005). Copper transporter-1 (Crt1) is a copper import protein that is copper-specific, and is believed to mediate copper uptake into the small intestine (Lee et al., 2002). Crt1 is expressed in the enterocytes of the small intestine and in enterocyte-like Caco-2 cells in culture (Klomp et al., 2002; Kuo et al., 2001). The copper efflux protein, ATP7A, is thought to mediate copper efflux across the plasma membrane during copper excess in transfected cells (Petris et al., 1996). Menkes disease, characterized by excessive copper accumulation in the intestine and systemic copper deficiency, is a consequence of a defect in ATP7A (Schaefer and Gitlin, 1999). ATP7B, with functional similarity to ATP7A, exports copper into bile for excretion (Roelofsen et al., 2000). ATP7B is localized primarily in the liver with lower expression found in the intestine, kidney and placenta (Lockhart et al., 2000). A defect in ATP7B results in Wilson's disease, characterized by copper toxicity (due to liver copper accumulation as a result of impaired biliary copper excretion) and liver damage.

### Metabolism and Excretion of Copper

The liver and intestine play key roles in copper metabolism. Copper is taken up by hepatocytes from the portal circulation. Inside the hepatocytes, copper is bound to metallothionein, a protein that also binds zinc, iron and mercury. Copper can be released from hepatocytes into the general circulation to be transported to other tissues, or it can be excreted from the liver in bile (Cousins, 1985). The major route of excretion is in the bile. Only a small amount is excreted in the urine (Cousins, 1985). Biliary excretion in human infants is immature at birth, and the lack of an effective excretion mechanism may place infants at increased risk for copper toxicity.

### Physiological/Nutritional Role of Copper

Because copper is an essential nutrient, an understanding of its numerous physiological roles in the body is essential for understanding the deleterious effects of copper deficiency or excess. Copper is essential for hemoglobin synthesis and erythropoiesis (Solomons, 1985; Harris, 1997). Copper deficiency can therefore lead to anemia. Copper deficiency can likewise lead to abnormalities of myelin formation, with attendant effects on the nervous system (Solomons, 1985; Harris, 1997). Nervous system effects, including dementia, have been observed in individuals with copper deficiency or excess (Solomons, 1985; Harris, 1997). Effects on catecholamine metabolism likewise are involved in the nervous system abnormalities. Other physiological functions that involve copper include leukopoiesis, skeletal mineralization, connective tissue synthesis, melanin synthesis, oxidative phosphorylation, thermal regulation, antioxidant protection, cholesterol metabolism, immune and cardiac function, and regulation of glucose metabolism. Since all of these physiological processes involve copper, any of them can be affected by the availability of copper in the body or in specific tissues. In general, deleterious effects may occur in any of these physiological processes due to either deficiency or excess of copper in the systems affected (Solomons, 1985; Harris, 1997).

The specific copper requirements of infants have not been well established. In infants, copper is an essential mineral that is required for normal growth, and the development of bone, brain, immune system, and red blood cells (Hurley et al., 1980). Full-term infants are believed to possess adequate copper stores at birth to last through weaning, but premature infants, prone to copper deficiency, must be given higher provisions of copper to compensate for inadequate copper stores (Lonnerdal, 1998).

Recommended Daily Allowances (RDAs) of copper were not provided in earlier RDA compilations because of difficulty in determining the values (NAS, 1989). Homeostatic mechanisms result in variable absorption and excretion of copper as dietary intake is manipulated, complicating mass balance calculations in dietary studies. However, in the most recent publication of recommended allowances (FNB, 2000), copper nutritional requirements have at last been established. Table 1 (below) shows the Dietary Reference Intake (DRI) values for copper for various age groups, broken down into Estimated Average Requirements (EAR), Recommended Dietary Allowances (RDA), and Tolerable Upper Intake Levels (UL) (FNB, 2000). Values for infants were provided only as Adequate Intake values, based primarily on the content of copper in human milk. The AI values are 200 μg/day for infants 0-6 months of age, and 220 μg/day for infants at 7-12 months; an estimated UL for infants could not be established (FNB, 2000).

TABLE 1
Recommended Daily Copper Dietary Reference Intakes by Sex/Age

| | | Estimated | Recommended | Tolerable |
|---|---|---|---|---|
| | | Average | Dietary | Upper Intake |
| Age (years) | Sex | Requirement | Allowance | Level |
| 1-3 | F/M | 260 | 340 | 1,000 |
| 4-8 | F/M | 340 | 440 | 3,000 |
| 9-13s | F/M | 540 | 700 | 5,000 |
| 14-18 | F/M | 685 | 890 | 8,000 |
| 18+ | F/M | 700 | 900 | 10,000 |
| Pregnant, 14-18 | F | 785 | 1,000 | 8,000 |
| 19+ | | 800 | 1,000 | 10,000 |
| Lactating, 14-18 | F | 985 | 1,300 | 8,000 |
| 19+ | | 1,000 | 1,300 | 10,000 |

Values from FNB, 2000.

Copper intake values from food and supplements, developed from the NHANES III nationwide survey (1988-1994), are shown in Table 2 (below). The NHANES III table and Continuing Survey of Food Intakes of Individuals (C SFII) indicate that intake of copper is adequate for the great majority of the population in all age and sex groups. However, results for some younger sex/age groups indicate as much as 10 percent of the population consuming less than the RDA of copper. On the other hand, considering the tendency for underreporting of food intakes, particularly for teenagers (Champagne et al., 1998), the lower end of the distribution curve is likely to be inaccurate.

TABLE 2
Copper Intake (mg/day) from Food and Supplements Versus
the Recommended Dietary Allowance (RDA)^{a}

| | | Percentile | | | | | | | | RDA |
|---|---|---|---|---|---|---|---|---|---|---|
| Age and Sex | | 5 | 10 | 25 | 50 | 75 | 90 | 95 | 99 | (mg/day) |
| 2-6 mo | M/F | 0.3 | 0.4 | 0.5 | 0.7 | 0.9 | 1.1 | 1.2 | 1.6 | 0.20 |
| 7-11 mo | M/F | 0.3 | 0.4 | 0.5 | 0.7 | 0.9 | 1.2 | 1.3 | 1.7 | 0.22 |
| 1-3 yr | M/F | 0.3 | 0.4 | 0.5 | 0.7 | 1.0 | 1.3 | 1.7 | 2.9 | 0.34 |
| 4-8 yr | M/F | 0.59 | 0.67 | 0.80 | 0.95 | 1.14 | 1.36 | 1.61 | 3.06 | 0.44 |
| 9-13 yr | F | 0.64 | 0.72 | 0.86 | 1.04 | 1.26 | 1.54 | 1.84 | 3.23 | 0.70 |
| | M | 0.88 | 0.94 | 1.05 | 1.21 | 1.41 | 1.61 | 1.78 | 3.13 | 0.70 |
| 14-18 yr | F | 0.64 | 0.75 | 0.89 | 1.08 | 1.32 | 1.64 | 1.96 | 3.32 | 0.89 |
| | M | 0.79 | 0.89 | 1.11 | 1.42 | 1.80 | 2.28 | 2.71 | 3.56 | 0.89 |
| 19-30 yr | F | 0.77 | 0.83 | 0.95 | 1.12 | 1.38 | 1.82 | 3.03 | 3.84 | 0.90 |
| | M | 1.37 | 1.43 | 1.56 | 1.69 | 1.86 | 2.12 | 3.55 | 4.44 | 0.90 |
| 31-50 yr | F | 0.72 | 0.81 | 0.95 | 1.17 | 1.52 | 2.32 | 3.09 | 4.19 | 0.90 |
| | M | 0.89 | 1.03 | 1.29 | 1.61 | 2.09 | 2.93 | 3.67 | 4.87 | 0.90 |
| 51-70 yr | F | 0.61 | 0.68 | 0.84 | 1.07 | 1.48 | 2.92 | 3.25 | 4.22 | 0.90 |
| | M | 0.75 | 0.87 | 1.09 | 1.43 | 1.98 | 3.00 | 3.65 | 5.02 | 0.90 |
| 71+ yr | F | 0.58 | 0.65 | 0.80 | 1.02 | 1.37 | 2.94 | 3.21 | 3.79 | 0.90 |
| | M | 0.72 | 0.83 | 0.99 | 1.26 | 1.66 | 2.89 | 3.41 | 4.61 | 0.90 |
| Pregnant | F | 0.71 | 0.82 | 1.07 | 1.62 | 3.11 | 4.03 | 4.39 | 5.56 | 1.0 |

^{a}Breast-feeding infants and children, and eight individuals reporting greater than 150 mg/day of copper from supplements excluded from the analysis.
RDA values from FNB, 2000.

While the majority of persons may be able to cope with chronic exposure to toxic copper ions contained in drinking water without showing signs of disease, there are certain rare diseases in which a person's copper transport and metabolic pathways are affected by genetic mutations, such as Wilson's disease and Menkes disease. The genetic mutations responsible for Wilson's disease and Menkes disease were identified for the first time in the 1990's by several groups. In addition to the rare Wilson's disease patients, there have been some published reports of elevated levels of serum copper in the elderly (Madaric et. al., Physiol Res, 1994; 43(2):107-11 and Ghayour-Mobarhan et. al., Ann Clin Biochem, 2005 September; 42(Pt 5):364-75), which may be attributable to a compromised ability to properly process and excrete copper via the liver, into the bile, and ultimately through the stool. Accordingly, this patient population may share a sensitivity to copper which is similar to that of Wilson's disease patients. Elevated levels of non-ceruloplasim bound copper have been reported in elderly Alzheimer's disease patients (Squitti, et. al.).

Wilson's disease is characterized by a mutation of the gene encoding the P-type ATPase, called ATP7B. Due to the impairment of ATP7B, Wilsons' disease patients are unable to adequately process, transport and excrete copper through the normal bile ducts of the liver. In the case of normal subjects, copper that is newly introduced is expected to first bind to available engogenous cuproproteins having the highest affinity for copper, such as metallothionein, superoxide dismutase and albumin. Free copper ions are relatively rare in serum, but copper which is “loosely bound” to various proteins and peptides can be substantial and elevated in Wilson's disease patients and potentially also in other metabolically compromised groups such as Alzheimer's disease patients, mild cognitive impairment (MCI) patients, schizophrenia patients, dementia patients, and the elderly.

In the 1990's, the genetic defect responsible for Menkes Disease was identified as a mutation to another P-type ATPase, ATP7A. Menkes Disease is characterized by abnormally low levels of available copper, due to the failure of intestinal cells to release copper, and results in various developmental abnormalities.

The following are estimated contributions to total serum copper content of various serum proteins in normal patients: ceruloplasmin (650-750 ug/L, 65-70%), albumin (120-180 ug/L, 12-18%), transcuprein (macroglobulin) (90 ug/L, 9%), ferroxidase II (10 ug/L, 1%), extracellular SOD and histidine rich glycoproteins (<10 ug/L, <1%), blood clotting factors V and VIII (<5 ug/L, <0.5%), extracellular metallothionein and anime oxidase (<1 ug/L, <0.1%), 15-60 kDa components (40 ug/L, 4%), small peptides and amino acids (35 ug/L, 4%), and, ultimately, unbound or “free” copper ions (0.0001 ug/L, approx. 0%) (Linder, M C Biochemistry of Copper (ed.) 1991; Linder, M C (2001), Copper and Genomic Stability in Mammals., Mutat. Res. 475, 151-52).

As noted, a substantial proportion of circulating serum copper is bound to 15-60 kDa proteins (approx. 4%) and small peptides and amino acids (an additional approximated 4%). Such small proteins and peptides are capable of transporting loosely bound copper across the blood brain barrier, thereby creating an environment wherein copper may exist in excess and may therefore be detrimental to the health of neurons. In order to maintain a healthy copper homeostasis and to protect from extracellular lipid peroxidation and intracellular oxidation, neurons may upregulate a variety of copper binding proteins, including APP, Amyloid beta, tau, BACE1 and apoE, all of which are upregulated in Alzheimer's disease (and intracellularly in a similar fashion in the neuromuscular disease, inclusion body myositis).

Solubilized copper or copper loosely bound to small ligands, such as that commonly found in tap water, is highly bioavailable (up to 65%) and, due to water fluxes in the intestines, has the capacity to overwhelm the copper homeostasis mechanisms of the gastrointestinal enterocytes and liver, and enter the portal and systemic circulation in a potentially toxic form loosely bound to albumin and other low kinetic copper binding proteins. It is an object of the present invention to provide compositions, formulations, agents and methods to protect the individual from such toxic fluxes, as further described herein.

### Wilson's Disease

In the case of untreated Wilson's disease patients, body copper continues to accumulate, ultimately overwhelming the high affinity cuproproteins. Residual copper either remains free and unbound or loosely bound to cuproproteins having low affinities to copper. This pool of free, unbound or loosely bound copper is free to circulate and may cross the blood brain barrier, damaging nerve cells due to its reactivity and pro-oxidant capacity. Clinically, the various cuproproteins serve as a reservoir for copper and will generally be released based upon an inverse correlation with each cuproprotein's individual affinity for copper. The so called “loosely bound” cuproproteins include albumin and homocysteine, for example. Such cuproproteins serve as potential toxic pools of available copper as compared to high affinity copper binding proteins, such as ceruloplasmin and cu/zn superoxide dismutase.

When the ability to excrete copper is impaired because of genetic disease or because there is hepatic impairment due to fibrosis or bile flow, the higher affinity cuproproteins, such as metallothionein, will ultimately become fully saturated and the copper binding process will continue with the lesser affinity copper binding proteins, such as homocysteine and albumin, until each of these protein pools become saturated as well. In the case of Wilson's patients, total copper overwhelms the capacity of high affinity cuproproteins to adequately bind and sequester it. The excess elevated body copper, so-called “free” or “unbound” or “loosely bound” copper, builds up in the body and is available to cross the blood brain barrier into the central nervous system. Over time, this copper toxicity damages or destroys organ systems such as the brain and liver.

### Treatment for Wilson's Disease

Where a network of cuproproteins is abnormal as a result of genetic abnormality or aging, for example, then copper in the body may not be properly bound and sequestered by cuproproteins and therefore may not be properly maintained. Toxicities and oxidative capacities are most pronounced when copper is present in the body in its so-called “free”, “unbound” or “loosely bound” forms. Such free copper can be toxic to various organ systems, such as the liver and the brain, for example. The classic example of such a disease is Wilson's disease. Treatment varies for patients that are initially presenting, and they are generally treated on an acute or induction basis, with potent copper chelators and complexors, such as tetrathiomolybdate, penicillamine or trientine, each of which is intended to either remove available free copper from the body or render it unavailable for further damage. Following initial acute or induction treatment, patients will be switched to a chronic or maintenance therapy on a long term basis, generally for the remainder of their lives. Agents commonly used for chronic or maintenance therapy include those that maintain a state of copper malabsorption, such as zinc acetate (Brewer). Zinc acetate is available as a prescription and is marketed in the United States under the tradename Galzin® and in Europe under the tradename Wilzin®. Other zinc salts available without a prescription in the United States have been reportedly used by Wilson's patients for long term maintenance therapy with varying degrees of success. Examples of such other salts include, but are not limited to, zinc carbonate, zinc sulfate, zinc gluconate, zinc oxide, zinc chloride and zinc stearate, for example.

Zinc has been for the comprehensive treatment of Wilson's disease including initial treatment (Hoogenraad et al., Lancet, 2:1262-1263 [1978]; Hoogenraad et al., Eur. Neurol., 18:205-211 [1979]; and Hoogenraad et al., J. Neurol. Sci., 77:137-146 [1987]). However, zinc was not ideal for initial therapy (by itself) because it is rather slow acting. Thus, it takes approximately two weeks to achieve intestinal metallothionein induction and a negative copper balance in Wilson's patients (Yuzbasiyan-Gurkan et al., J. Lab. Clin. Med., 120:380-386). [1992]). At the two week point, zinc immediately reverses the +0.54 mg daily (positive) copper balance that these patients average, but the negative copper balance induced is rather modest, averaging −0.35 mg daily (negative) copper balance (G J Brewer et al., J. Trace Elem. Exp. Med., 3:227-234 [1990]; G J Brewer et al., Amer. J. Med. Sci., 305:(4)199-202 [1993]). Due to this low rate of copper removal, it takes as long as six months of zinc therapy to bring urine copper and nonceruloplasmin plasma copper (the potentially toxic copper measured in the blood), down to subtoxic levels. Tetrathiomolybdate (TM) is a more effective blocker of copper absorption than zinc, since zinc acts only in those areas of the small intestine where metallotionein can be induced. In contrast, TM works all up and down the gastrointestinal track. The other advantage of TM over zinc is that TM acts immediately. It does not have a lag period required for the induction of metallothionein.

Ultimately, such chronic maintenance therapies fail in some patients due to chronic and acute stomach and esophageal irritation and nausea commonly associated with such agents, difficulty in predicting effects and in setting an appropriate dosing regimen, and the need to continuously monitor free serum copper levels in order to assure that they are maintained within the normal range. The variability of effect of such agents depends upon the timing of administration as it relates to the timing of meals, difficulties in maintaining adequate patient compliance given the daily multiple dosing regimen, possible stomach irritation and the need to time each dose at least one hour prior and three hours after meals, as well as the need to assure compliance for the entire remaining lifetime of the patient.

Other Disorders Associated with Elevated Levels of Free Copper and/or Accumulation of Copper

Other disorders are associated with elevated levels of loosely bound, free copper, and/or accummulation of copper includes headaches, hypoglycemia, increased heart rate, nausea, anemia, hair loss, nephritis, autism, depression, hallucinations, hyperactivity, insomnia, disperception of the senses, paranoia, personality changes, psychosis, schizophrenia, mild cognitive impairment, detachment from reality, atherosclerosis, stroke, tauapathies and synucleinopathies, nonalcoholic steatohepatitis, multiple sclerosis, Alzheimer's, Parkinson's, dementia, ALS and autism which are given as exemplary. In addition, diseases associated with increased inflammation and fibrosis are associated with normal to elevated levels of free copper and can be alleviated by a reduction of these levels via copper reduction intervention.

### Alzheimer's Disease

As human life span has significantly expanded over the last century, Alzheimer's disease and other neurodegenerative diseases will have a growing impact on the quality of life for a large proportion of the population. For example, Alzheimer's disease is a leading cause of dementia in the elderly, affecting 5-10% of the population over the age of 65 years. See A Guide to Understanding Alzheimer's disease and Related Disorders, edited by Jorm, New York University Press, New York (1987). Alzheimer's disease often presents with a subtle onset of memory loss followed by a slow progressive dementia over several years. The prevalence of Alzheimer's disease and other dementias doubles every five years beyond the age of 65. See 1997 Progress Report on Alzheimer's disease, National Institute on Aging/National Institute of

Health. Alzheimer's disease now affects 12 million people around the world, and it is projected to increase to 22 million by 2025 and to 45 million by 2050. See Alzheimer's Association Press Release, Jul. 18, 2000.

The complexity of the brain's architecture and chemistry, and the complexity of these neurodegenerative brain diseases, especially Alzheimer's disease, has hampered the development of a model that mimics many of the changes seen in the human brain. Such a model is needed in order to identify drugs or other agents that might be useful in treating, preventing or reversing the effects of such diseases.

Alzheimer's disease is histopathologically characterized by the loss of particular groups of neurons and the appearance of two principal lesions within the brain, termed senile plaques and neurofibrillary tangles. See Brion et al., J. Neurochem. 60:1372-1382 (1993). Senile plaques occur in the extracellular space. A major component of senile plaques is beta-amyloid (A-beta), a naturally secreted but insoluble peptide formed by cleavage of amyloid precursor protein (APP). A-beta is a fragment close to the carboxyterminal domain of APP.

Neurofibrillary tangles are intraneuronal accumulations of filamentous material in the form of loops, coils or tangled masses. They are most abundantly present in parts of the brain associated with memory functions, such as the hippocampus and adjacent parts of the temporal lobe. See Robbins Pathologic Basis of Disease, Cotran et al., 6.sup.th ed. (1999). Neurofibrillary tangles are commonly found in cortical neurons, especially in the entorhinal cortex, as well as in other locations such as pyramidal cells of the hippocampus, the amygdala, the basal forebrain, and the raphe nuclei.

Neurofibrillary tangles can also be found during normal aging of the brain, however, they are found in a significantly higher density in the brain of Alzheimer's disease patients, and in the brains of patients with other neurodegenerative diseases, such as progressive supranuclear palsy, postencephaltic Parkinson disease, Pick's disease, amylotrophic lateral sclerosis, etc. Robbins Pathologic Basis of Disease, Cotran et al., 6th ed. (1999), p. 1330. Previous studies suggest that, among other things, neurofibrillary tangles may significantly contribute to the cognitive decline associated with the disease and also directly to neuronal cell death.

Ultrastructurally, neurofibrillary tangles are composed predominantly of paired helical filaments (“PHF”). A major component of PHF is an abnormally phosphorylated form of a protein called tau and its fragments. Robbins Pathologic Basis of Disease, Cotran et al., 6th ed., W. B. Saunders Company (1999), p. 1300.

The tau protein (also referred to as “native tau”) is a microtubule-associated phosphoprotein that stabilizes the cytoskeleton and contributes to determining neuronal shape. See Kosik & Caceres, Cell Sci. Suppl. 14:69-74 (1991). Tau has an apparent molecular weight of about 55 kDa. The protease cathepsin D cleaves tau protein at neutral (cytoplasmic) pH resulting in tau fragments—one of which has a molecular weight of approximately 29 kDa (referred to by some authors as “tau fragment”). See, e.g., Bednarski & Lynch, J. Neurochem. 67:1846-1855 (1996); Bednarski & Lynch, NeuroReport 9:2089-2094 (1998). Both the tau protein and 29 kDa tau fragment can be phosphorylated. In a normal brain, the tau protein and tau fragment typically exist in an unphosphorylated, or dephosphorylated state. However, in neurofibrillary tangles, both tau protein and tau fragment can be found in an abnormally phosphorylated state, a hyperphosphorylated state. The 29 kDa tau fragment is a major component of neurofibrillary tangles. Hyperphosphorylation impairs tau protein's ability to interact with microtubules.

Bednarski E, and Lynch G, J Neurochem 67:1846-55 (1996) cultured hippocampal slices with an inhibitor [N-CBZ-L-phenylalanyl-L-alanine-diazomethyl ketone (ZPAD)] of cathepsins B and L. The authors reported that this resulted in the degradation of high molecular weight isoforms of tau protein and the production of a 29-kDa tau fragment (tau 29).

Bednarski E, and Lynch G, Neuroreport 9:2089-2094 (1998) reported that incubating cultured hippocampal slices with chloroquine or with ZPAD resulted in increases in enzymatically active cathepsin D and the delayed appearance of a 29 kDa fragment of the tau protein. The authors proposed that inactivation of cathepsin L leads to induction of cathepsin D which leads to aberrant tau proteolysis and that such a pathway is likely to play an important role in brain aging.

In addition to the build-up of A-beta and of neurofibrillary tangles, increasing evidence has pointed to a link between lipid metabolism and Alzheimer's disease. Epidemiological studies found that patients with increased plasma low density lipoprotan cholesterol and cholesterol levels and cardiovascular diseases have an increased risk of Alzheimer's disease Kuo, Y-M, et al., Biochem. Biophys. Res. Comm. 252: 711-715 (1998); Jick, H., et al., Lancet 356:627-631 (2000). Also, long-term therapy with the 3-hydroxy-3-methylglutaryl coenzyme A reductase inhibitors appears to decrease the prevalence of Alzheimer's disease (Jick, H., et al., Lancet 356:627-631 (2000); Wolozin, B., et al., Arch. Neurol. 57:1439-1443 (2000)).

Consistent with a link to lipid metabolism, in vitro experiments have shown that cholesterol affects the generation and aggregation of beta amyloid (A-beta) (Bodovitz, S., and Klein, W. L., J. Biol. Chem. 271:4436-4440 (1996); Xu, H., et al., Proc. Natl. Acad. Sci. USA 94:3748-3752 (1997); Howland, D. S., et al., J. Biol. Chem. 273:16576-16582 (1998)). Transgenic mice fed a high cholesterol diet also developed increased amounts of A-beta deposition (Refolo, L. M., et al., Neurobiol. Dis. 7:321-331 (2000)).

ApoB and apoE mediated transport of cholesterol into lysosomes is a critical step for cells to utilize these sterols, which is of particular importance for mature neurons that mainly rely on extracellular apoE mediated transport of cholesterol (Brown, M. S., and Goldstein, J. L., Annu. Rev. Biochem. 52:223-261 (1983)). Once in the lysosome, cholesterol and other lipids dissociate from ApoE before being utilized by the cell (Brown, M. S., and Goldstein, J. L., Annu. Rev. Biochem. 52:223-261 (1983)).

Changes in cholesterol levels may be involved in certain neurodegenerative diseases. For example, accumulation of insoluble A-beta1-42 has been found in Niemann-Pick type C (NPC) mutant cells (Yamazaki, T., et al., J. Biol. Chem. (2000)). These cells exhibit many pathologic characteristics, one of which is impaired intracellular transport of cholesterol (Millard, E. E., et al., J. Biol. Chem. 275:38445-38451 (2000)). Also, the ApoE4 isoform is a known risk factor for late-onset Alzheimer's disease.

Inhibition of cholesterol synthesis enhanced the phosphorylation of tau in dissociated cell cultures [ref. in (Sawamura, N., et al., J. Biol. Chem. 57:1439-1443 (2001))]. Likewise, hyperphosphorylation of tau has been demonstrated in cell cultures prepared from NPC mutant mice (Sawamura, N., et al., J. Biol. Chem. 57:1439-1443 (2001)). Gradually developing disturbances in lysosomes, which affect the sorting/trafficking of cholesterol from lysosomes and late endosomes, may, therefore, be contributors to the pathologies associated with neurodegenerative diseases and Alzheimer's disease.

U.S. Pat. No. 6,803,233 describes animal models of Alzheimer's disease in which cysteine protease inhibitors are capable of producing animal models of Alzheimer's disease including the hallmark neurofibrillary tangles (NFTs), composed of paired helical filaments of tau are concentrated. Such patent, however, does not describe the copper binding effects of cysteine nor homocysteine as it to the creation of available pools of low molecular weight copper-cysteine complexes capable of crossing the blood brain barrier and upregulating the production of APP, Aβ and tau proteins. In one aspect, the present invention involves formulations of zinc (and more preferentially, gastroretentive sustained release zinc) and folic acid to reduce and stabilize the systemic and CSF levels of low molecular weight copper cysteine complexes (such as copper-homocysteine) that the present inventors recognize as a contributing factor to neurodegenerative diseases such as Alzheimer's disease, Parkinson's disease and ALS, for example.

### Prior Art Involvement of Copper in Alzheimer's Disease, Parkinson's Disease, ALS and Other Disorders of the Central Nervous System (CNS).

At present in the prior art, there is a considerable amount of conflicting conclusions and hypotheses regarding the causative role of elemental copper and zinc in neurodegenerative diseases, such as Alzheimer's disease, Parkinson's disease, ALS and CJD. For example, it is known that many of the hallmark proteins associated with Alzheimer's disease are copper binding proteins, including amyloid precursor protein (APP), beta amyloid (Aβ) (including peptides 1-40 and 1-42), tau (including the paired helical fragments (PHFs) and neurofibrillary tangle (NFTs)), beta secretase (BACE1) and apolipoprotein E (apoE), including the three major human variants of apoE, apoE2, apoE3 and apoE4. Regarding the latter, it is noted by the inventors that apoE2, apoE3 and apoE4 differ only in regard to the presence or absence of cysteine residues at positions 112 and 158. it has previously been shown that apoE2, apoE3 and apoE4 differ in their ability to bind copper.

Epidemiological genetic studies indicate that presence of the apoE4 varient, having no cysteine residues at positions 112 and 158, increases the risk of AD, while apoE2, having two cysteine residues at positions 112 and 158, is considered to have protective benefit for Alzheimer's disease (as well as athlerosclerosis) as compared to the most common hum varient apoE3, which has only cysteine residue these positions.

Pursuant to published findings of the Framingham study, elevated levels of homocysteine have been implicated with an increased risk of Alzheimer's disease, although until the disclosure contained herein, the effects homocysteine as a low molecular weight copper binding protein capable of delivering, maintaining and slowing clearance of toxic, loosely bound, and therefore exchangable “free” copper in the CNS has not been previously described.

In addition, elevated levels of cholesterol have been implicated with an increased risk of Alzheimer's disease. In particular, elevated levels of oxidized cholesterol, 27S-hydroxy-cholesterol and/or 24S-hydroxy-cholesterol, have been found both in the CNS and circulation and circulation of Alzheimer's patients (as well athlerosclerosis). In addition to Alzheimer's disease, elemental copper has also been hypothesized to play a role in other neurodegenerative diseases, such as, ALS, in which an mutant form of the copper/zinc binding protein, Cu/Zn superoxide dismutase (SOD1) has reduced ability to bind copper.

In Parkinson's disease, the copper, iron and aluminum binding protein, α-synuclein (AS) is known to be the major component of the neuronal and glial cytoplasmic inclusions known as Lewy Bodies widely considered as the hallmark lesions of both Parkinson's disease as well as the group of neurodegenerative disorders referred to as synucleinopathies.

### Neural Tube Defects

Neural tube defects (NTDs) are major birth defects of the fetal brain or spine, and occurs when the neural tube (that later turns into the brain and spine) doesn't properly form, resulting in brain or spine damage. This occurs within the first few weeks a woman is pregnant, often before a woman knows that she is pregnant. Adequate intake of the B vitamin, folic acid by mothers prior to pregnancy has been shown to reduce the incidence of NTDs by up to 70% although the mechanism by which folic acid exerts this benefit has not yet been previously described. CDC, Folic Acid Now, CDC-NCEH99-0463, November 2005.

Spina bifida and anencephaly are two common types of NTDs. About 3,000 pregnancies in the United States are affected by spina bifida or anencephaly each year. Spina bifida occurs when the spine and back bones do not close all the way. When this happens, the spinal cord and back bones do not form as they should. A sac of fluid comes through an opening in the baby's back. Much of the time, part of the spinal cord is in this sac and it is damaged. Most children born with spina bifida live full lives, but they often have lifelong disabilities and need many surgeries.

Children born with spina bifida don't all have the same needs. Some children's problems are much more severe than others. Even so, with the right care, most of these children will grow up to lead full and productive lives.

Anencephaly occurs when the brain and skull bones do not form right. When this happens, part or all of the brain and skull bones might be missing. Babies with this defect die before birth (miscarriage) or shortly after birth.

Folic acid might help to prevent some other birth defects, such as cleft lip and palate and some heart defects. There might also be other health benefits of taking folic acid for both women and men. Low zinc and high levels of copper have been found in mothers of children with isolated cleft lip and palate. Hoyasz K K, Wiad. Lek., 58(7-8):382-5 (2005). Until reported in U.S. patent application Ser. No. 11/621,962, filed 10 Jan. 2007, and published on 6 Sep. 2007 as patent publication no. US2007/0207191 A1, and International Patent Application No. PCT/US2007/060345, filed 10 Jan. 2007, and published on 26 Jul. 2007 as patent publication no. WO 2007/084818 A2, the role of folic acid in reducing pool of circulating serum copper bound to homocysteine has not been previously described.

### Antioxidants

### Acetylcholine Esterase Inhibitors

Acetylcholinesterase inhibitors are highly regarded clinical agents for treating and improving senile dementia such as Alzheimer type senile dementia, or cerebrovascular dementia, attention deficit hyperactivity disorder and schizophrenia. In particular, donepezil hydrochloride (1-benzyl-4-[(5,6-dimethoxy-1-indanone)-2-yl]methylpiperidine hydrochloride) has been found to be useful as a acetylcholinesterase inhibitor in providing a desired pharmacological activity with minimum adverse side effects. In addition to donepezil hydrochloride, other known acetylcholinesterase inhibitors include rivastigmine (3-[1-(dimethylamino)ethyl]phenyl N-ethyl-N-methylcarbamate), metrifonate (dimethyl 2,2,2-trichloro-1-hydroxyethyl)phosphate), tacrine hydrochloride (1,2,3,4-tetrahydro-9-acridinamine), galanthamine hydrobromide, neostigmine, physostigmine etc. An object of the present invention as further described herein includes formulations that combine acetyl-cholinesterase inhibitors with agents selected from the group of zinc, zinc-cysteine tetrathiomolybdate, gastroretentive sustained release zinc formulations and sustained release formulations of other essential trace metals such as, copper and iron.

Zinc is also an important anti-oxidant. However, most adults are zinc deficient, and taking high dose zinc requires copper add back to avoid copper deficiency. Zinc also upsets the stomach. Thus, zinc normally is injected in an enteric coated, delayed release formulations.

Most nutritional supplements that contain copper or iron contain the copper or iron as pure salts. However, when supplied is pure salts, the copper and iron may enter the blood too quickly, resulting in elevated free copper and iron in the blood, which in turn may result in problems as discussed above.

Additionally, Morris et al. in Arch. Neurol. 2006; 63:1085-1088 report cognitive decline for people over age 65 who took copper-containing supplements with a high fat diet.

In U.S. patent application Ser. No. 11/621,962, filed 10 Jan. 2007, and published on 6 Sep. 2007 as patent publication no. US2007/0207191 A1, and International Patent Application No. PCT/US2007/060345, filed 10 Jan. 2007, and published on 26 Jul. 2007 as patent publication no. WO 2007/084818 A2 the present inventor describes certain sustained release copper, zinc, iron and other trace metal formulations for addressing copper, iron and trace metal intake.

Incorporated herein by reference are the following patent publications listing the present inventor as an inventor: WO 2007/092966 A2, WO 2007/092966 A3, and US2007/209950 A1.

### BRIEF SUMMARY OF THE INVENTION

The present invention provides improved nutritional supplements containing, for example, bound copper or bound iron or bound copper and bound iron alone or with zinc in slow release form.

The present invention includes a multi-vitamin preparation containing delayed or sustained release bound copper. Preferably, the multi-vitamin preparation also contains delayed or sustained release bound iron.

The present invention includes a multi-vitamin preparation containing delayed or sustained release zinc.

The present invention includes a multi-vitamin preparation containing delayed or sustained release bound copper and sustained released bound iron.

The present invention includes a multi-vitamin preparation containing delayed or sustained release bound copper and delayed or sustained released zinc.

The present invention includes a multi-vitamin preparation containing delayed or sustained release bound copper, delayed or sustained release bound iron, and delayed or sustained release zinc.

The present invention includes a multi-vitamin preparation containing delayed or sustained release bound iron and delayed or sustained release zinc.

The delayed or sustained release copper, delayed or sustained release iron and/or delayed or sustained release zinc are preferably enteric coated.

The delayed or sustained release copper, delayed or sustained release iron and/or delayed or sustained release zinc are preferably bound to a pharmaceutically acceptable, stable, natural or synthetic carrier. Preferably, the carrier comprises a plant fiber.

The copper, iron and/or zinc are preferably bound to low molecular weight amino acid. The copper, iron and/or zinc can be dissolved in a solution of dried or evaporated milk, dried whey, dried milk lipids and dried milk proteins.

The zinc preferably comprises a zinc cysteine complex.

The zinc can comprise, for example, 25-75 mg delayed or sustained release zinc.

The preparation can be in the form of a pill or tablet.

The preparation can also include delayed or sustained release molybdenum.

The preparation can also include delayed or sustained release sulfur.

The preparation can also include delayed or sustained release molybdenum and sulfur.

The preparation can comprise the components of multi-vitamins and in the amounts detailed in the Harvard Food Frequency Questionnaire without any immediate release or free or inorganic copper.

Preferably, redox active minerals including copper or iron are complexed with digestible protein, fiber, or other natural or synthetic material so as to minimize the potential bolus flux of such metals into the serum of patients.

Preferably, redox active minerals including copper or iron are in a sustained release form so as to minimize the potential bolus flux of such metals in a free form into the serum of patients.

The present invention also includes a multi-vitamin preparation containing delayed or sustained release copper and zinc in a substantially immediately available form. The copper is preferably complexed with digestible protein, fiber, or other natural or synthetic material so as to minimize the potential bolus flux of such copper into the serum of patients. The zinc is preferably selected from the group of zinc, a zinc-cysteine complex, zinc acetate or another zinc salt; more preferably, the zinc is a zinc-monocysteine complex. Preferably, the zinc is in an immediately available form. For example, the zinc can be bioavailable within 20 minutes after oral ingestion away from food by a human. Preferably, the delayed or sustained release copper is not bioavailable until at least 60 minutes after oral ingestion away from food by a human. Preferably, the zinc is bioavailable in the jejunum and duodenum of a human after oral ingestion by the human of the preparation. Preferably, the delayed or sustained release copper is not bioavailable in the jejunum and duodenum of a human after oral ingestion by the human of the preparation. Preferably, the delayed or sustained release copper is not bioavailable until the delayed or sustained release copper reaches the duodenum of a human after oral ingestion by the human of the preparation.

The preparation can contain between about 7.5 and about 200 mg bioavailable elemental zinc and between about 0.45 and about 8 mg bioavailable elemental copper.

The preparation can contain between about 16.5 and about 100 mg bioavailable elemental zinc and between about 0.725 and about 5 mg bioavailable elemental copper.

The preparation can contain between about 25 and about 50 mg bioavailable elemental zinc and between about 1 and about 2 mg bioavailable elemental copper.

The preparation can comprise the components of any commercially available multi-vitamin supplement without any immediate release copper or free copper or inorganic copper.

The present invention also includes an oral mineral preparation containing an immediate release or delayed release zinc moiety together with sustained release copper. Preferably, the mineral preparation is enteric coated. Preferably, the zinc moiety is in an inorganic form and the copper is in a bioinorganic form. The oral mineral preparation can contain, for example, 7.5 mg to 200 mg of elemental zinc. The oral mineral preparation can contain, for example, 1 mg to 8 mg of elemental copper The oral mineral preparation can contain vitamins as well.

The present invention also includes an oral mineral preparation containing an immediate release or delayed release zinc moiety together with sustained release iron. The mineral preparation is preferably enteric coated. Preferably, the zinc moiety is in an inorganic form and the iron is in a bioinorganic form. The oral mineral preparation can contain, for example, 7.5 mg to 200 mg of elemental zinc. The oral mineral preparation can contain, for example, 3 mg to 36 mg of elemental iron, preferably 6-24 mg, and more preferably 9-18 mg. The oral mineral preparation can contain vitamins as well.

The present invention includes a multimineral dietary supplement composition for oral administration containing, per unit dose:
(a) an outer layer having a zinc in a coating that is a quicker release portion that is adapted to be released in the upper gastrointestinal tract, and
(b) a slow release copper core component, present in controlled release form, so adapted so as to be subsequently released in a controlled manner lower in the gastrointestinal tract. The formulation can be, for example, in the form of a tablet. The tablet can also contain a protective film coating surrounding said outer layer of one or both components. Component (a) can contain, for example, between about 7.5 and about 200 mg bioavailable zinc. Component (b) can contain, for example, between about 1 and about 8 mg bioavailable copper. Preferably, the zinc is in the form of zinc acetate, zinc sulphate or zinc monocysteine. Preferably, the copper is in the form of copper bound to protein. The multimineral composition preferably further comprises bioavailable magnesium in the form of magnesium oxide, magnesium hydroxide or magnesium sulfate. The zinc and copper can be in a laminated tablet.

One can treat a patient in need of treatment for excess free copper and for limiting the patient's exposure to free copper by administering to the patient the preparation of a preferred embodiment of the present invention of any prior claim. Preferably, the administration occurs daily or twice a day. Preferably, the patient is advised that the administration will help treat excess free copper.

### DETAILED DESCRIPTION OF THE INVENTION

More particularly, the present invention relates to administration to humans and animals multi-vitamins and trace elements including copper or iron or copper and iron, alone or with zinc in sustained or delayed release form. In one aspect, the present invention provides a food supplement in the form of a multi-vitamin that is either copper and iron free, or contains sustained or delayed release copper and/or sustained or delayed release iron alone or with sustained or delayed release zinc or a zinc cysteine complex and optionally including sustained or delayed release molybdenum and/or sulfur.

Multi-vitamins and supplements are available commercially include copper, iron and/or zinc in salt form. However, as noted supra, when supplied as pure salts, copper and iron may enter the blood too quickly, resulting in elevated free copper and iron which may cross the blood/brain barrier, while zinc may upset the stomach and deplete copper. The present invention provides multi-vitamin and food supplements with trace amounts of copper, iron and/or zinc in a delayed or sustained release formulation that avoids problems including gastrointestinal upset, metal depletion and, in the case of people with impaired liver function and the elderly, increased blood level and consequent possibility of crossing the blood/brain barrier.

Broadly, the present invention provides multi-vitamin or supplements containing a gastro retentive and/or delayed or sustained release pharmacological formulation incorporating one or more trace metals of zinc, copper and iron. The zinc, copper and/or iron may be formulated in a sustained or delayed release or gastro retentive form following the teachings of U.S. patent application Ser. No. 11/621,962, filed 10 Jan. 2007, and published on 6 Sep. 2007 as patent publication no. US2007/0207191 A1, and International Patent Application No. PCT/US2007/060345, filed 10 Jan. 2007, and published on 26 Jul. 2007 as patent publication no. WO 2007/084818 A2. For example, the copper, iron and/or zinc may be enteric coated. A typical enteric coating may be a polymeric material. Preferred enteric coating materials comprise bioerodible, gradually hydrolysable and/or gradually water-soluble polymers. The “coating weight,” or relative amount of coating material per capsule, generally dictates the time interval between ingestion and drug release. Any coating should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the practice of the present invention to achieve delivery of the active drug to the lower gastrointestinal tract. The selection of the specific enteric coating material depends on the following properties: resistance to dissolution and disintegration in the stomach; impermeability to gastric fluids and drug/carrier/enzyme while in the stomach; ability to dissolve or disintegrate rapidly at the target intestine site; physical and chemical stability during storage; non-toxicity; ease of application as a coating (substrate friendly); and economical practicality.

Suitable enteric coating materials include, but are not limited to: cellulosic polymers such as hydroxypropyl cellulose, hydroxyethyl cellulose, hydroxypropyl methyl cellulose, methyl cellulose, ethyl cellulose, cellulose acetate, cellulose acetate phthalate, cellulose acetate trimellitate, hydroxypropylmethyl cellulose phthalate, hydroxypropyhnethyl cellulose succinate and carboxymethylcellulose sodium; acrylic acid polymers and copolymers, preferably formed from acrylic acid, methacrylic acid, methyl acrylate, ammonio methylacrylate, ethyl acrylate, methyl methacrylate and/or ethyl methacrylate (e.g., those copolymers sold under the tradename “Eudragit”); vinyl polymers and copolymers such as polyvinyl pyrrolidone, polyvinyl acetate, polyvinylacetate phthalate, vinylacetate crotonic acid copolymer, and ethylene-vinyl acetate copolymers; and shellac (purified lac). Combinations of different coating materials may also be used to coat a single capsule. Particularly preferred enteric coating materials for use herein are those acrylic acid polymers and copolymers available under the tradename “Eudragit” from Rohm Pharma (Germany). The Eudragit series E, L, S, RL, RS and NE copolymers are available as solubilized in organic solvent, as an aqueous dispersion, or as a dry powder. The Eudragit series RL, NE, and RS copolymers are insoluble in the gastrointestinal tract but are permeable and are used primarily for extended release. The Eudragit series E copolymers dissolve in the stomach. The Eudragit series L, L-30D and S copolymers are insoluble in stomach and dissolve in the intestine, and are thus most preferred herein.

A particularly suitable methacrylic copolymer is Eudragit L, particularly L-30D and Eudragit 100-55. In Eudragit L-30D, the ratio of free carboxyl groups to ester groups is approximately 1:1. Further, the copolymer is known to be insoluble in gastrointestinal fluids having pH below 5.5, generally 1.5-5.5, i.e., the pH generally present in the fluid of the upper gastrointestinal tract, but readily soluble or partially soluble at pH above 5.5, i.e., the pH generally present in the fluid of lower gastrointestinal tract. Another particularly suitable methacrylic acid polymer is Eudragit S, which differs from Eudragit L-30D in that the ratio of free carboxyl groups to ester groups is approximately 1:2. Eudragit S is insoluble at pH below 5.5, but unlike Eudragit L-30D, is poorly soluble in gastrointestinal fluids having a pH in the range of 5.5 to 7.0, such as in the small intestine. This copolymer is soluble at pH 7.0 and above, i.e., the pH generally found in the colon. Eudragit S can be used alone as a coating to provide drug delivery in the large intestine. Alternatively, Eudragit S, being poorly soluble in intestinal fluids below pH 7, can be used in combination with Eudragit L-30D, soluble in intestinal fluids above pH 5.5, in order to provide a delayed release composition which can be formulated to deliver the active agent to various segments of the intestinal tract. The more Eudragit L-30D used, the more proximal release and delivery begins, and the more Eudragit S used, the more distal release and delivery begins. It will be appreciated by those skilled in the art that both Eudragit L-30D and Eudragit S can be replaced with other pharmaceutically acceptable polymers having similar pH solubility characteristics.

The enteric coating provides for controlled release of the active agent, such that active agent release can be accomplished at some generally predictable location in the lower intestinal tract below the point at which active agent release would occur without the enteric coating. The enteric coating also prevents exposure of the hydrophilic therapeutic agent and carrier to the epithelial and mucosal tissue of the buccal cavity, pharynx, esophagus, and stomach, and to the enzymes associated with these tissues. The enteric coating therefore helps to protect the active agent and a patient's internal tissue from any adverse event prior to drug release at the desired site of delivery. Furthermore, the coated capsules of the present invention allow optimization of drug absorption, active agent protection, and safety. Multiple enteric coatings targeted to release the active agent at various regions in the lower gastrointestinal tract would enable even more effective and sustained improved delivery throughout the lower gastrointestinal tract.

The coating can, and usually does, contain a plasticizer to prevent the formation of pores and cracks that would permit the penetration of the gastric fluids. Suitable plasticizers include, but are not limited to, triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, a coating comprised of an anionic carboxylic acrylic polymer will usually contain approximately 10% to 25% by weight of a plasticizer, particularly dibutyl phthalate, polyethylene glycol, triethyl citrate and triacetin. The coating can also contain other coating excipients such as detackifiers, antifoaming agents, lubricants (e.g., magnesium stearate), and stabilizers (e.g., hydroxypropylcellulose, acids and bases) to solubilize or disperse the coating material, and to improve coating performance and the coated product.

The coating can be applied to the capsule using conventional coating methods and equipment. For example, an enteric coating can be applied to a capsule using a coating pan, an airless spray technique, fluidized bed coating equipment, or the like. Detailed information concerning materials, equipment and processes for preparing coated dosage forms may be found in Pharmaceutical Dosage Forms: Tablets, eds. Lieberman et al. (New York: Marcel Dekker, Inc., 1989), and in Ansel et al., Pharmaceutical Dosage Forms and Drug Delivery Systems, 6.sup.th Ed. (Media, Pa.: Williams & Wilkins, 1995). The coating thickness, as noted above, must be sufficient to ensure that the oral dosage form remains intact until the desired site of topical delivery in the lower intestinal tract is reached.

While it is possible that a sustained release trace metal may be incorporated into sustained release pharmaceutically acceptable sustained release microsphere, matrix, pellet or particle (all of which are commonly known in the art) in the form as a pure cation or salt, it is preferable to first bind the trace metal to pharmaceutically acceptable, stable, natural or synthetic carriers to which such metals are known to bind, such as, for example, plant fiber, whey, metallotheionein, transferrin, proteins and/or milk or milk by-products. Such carriers will have the benefit of further inducing the gradual digestion and absorption of the trace metals as they are naturally found in foods. The invention thus further provides a gastroretentive and/or sustained release pharmaceutical formulation incorporating one or more trace metals, such as zinc, copper and iron together with one or more pharmaceutically acceptable carriers and, optionally, other therapeutic and/or prophylactic ingredients. The carrier(s) must be “acceptable” in the sense of being compatible with the other ingredients of the formulation and not deleterious to the recipient thereof.

In one embodiment of the invention a copper supplement is administered in conjunction with, or combined with, a copper malabsorption agent, wherein the copper is in a form bound to or formulated with lipids, whey or casein. A copper supplement formulated in such a fashion would be intended to mimic, in pill or capsule form, the manner in which copper is provided from mother to infant via human breast milk. It would therefore be expected that such copper, upon reaching the stomach and small intestines, would be appropriately processed in a proper digestive manner, as opposed to copper which is in water or in a pure salt form. It is anticipated that the processing of such a copper supplement would mimic the high bioavailability found in breast milk (24%) or cow milk (18%), while also permitting the normal digestion and processing of copper by the intestines and liver, thereby reducing the level of burden of free or loosely bound copper in the serum and CNS. Such formulations could also include other essential metals and minerals such as iron or zinc. In a preferred embodiment of the invention, a copper and/or iron supplement formulated with lipids, whey and other proteins, with which copper is normally found in breast milk, may be formulated with a copper malabsorption agent such as zinc so as to simultaneously provide a bioavailable amount of copper and/or iron in a form for normal processing by the intestines, while at the same time inducing the production of metallothionein in the intestines to block and protect against the subsequent absorption of ionic copper from drinking water. In a preferred embodiment, such carrier bound copper supplement is incorporated within a sustained release microparticle or matrix so as to further regulate the absorption, and reduce the potential to cause peak elevated levels of free copper in the systemic circulation and CSF. Such formulations should reduce or avoid the need to monitor patients for hypocupremia or anemia, and also lower the levels of free or loosely bound serum or CNS copper while bolstering the levels of ceruloplasmin bound copper (given its processing by the liver by virtue of the first pass effect and normal copper handling, which mimics that of the evolutionarily proven copper and metal supplementation methods by which a mother processes and passes nutritional copper and other metals to a newborn baby via breast milk). Such formulation could also include other essential trace elements in a carrier bound complexed sustained release formulation, such as, iron, molybdenum, and sulfur, for example.

The zinc and copper formulations described herein may also be administered with certain copper absorption enhancing agents such as glycerol and NaCl, or gum arabic, to increase the bioavailability of complexed copper.

The present invention preferably is administered as a single pill combined with multi-vitamins and minerals, but also could be administered as a two-pill system whereby complexed copper pills or formulations are orally administered first and are followed by orally administered zinc-containing pills either together or after sufficient delay.

The present invention also contemplates incorporation of other essential minerals for which intestinal zinc may also reduce bioavailability, such as iron, molybdenum and sulfur.

In a preferred embodiment of the invention, sustained release zinc is formulated using gastro retentive forms of zinc, enteric coated zinc and/or sustained release zinc such that the copper bound complexes are released into the gastrointestinal tract ahead of the zinc as explained in Example 11 of U.S. patent application Ser. No. 11/621,962, filed 10 Jan. 2007, and published on 6 Sep. 2007 as patent publication no. US2007/0207191 A1, and International Patent Application No. PCT/US2007/060345, filed 10 Jan. 2007, and published on 26 Jul. 2007 as patent publication no. WO 2007/084818 A2. A preferred amino acid is cysteine which complexes readily with zinc to form zinc cysteine which is then slowly released in the gastrointestinal tract.

An embodiment of the present invention is useful for protecting a person from daily exposure to free inorganic copper, such as, that commonly present in tap water having leached from the copper plumbing providing water. Oral immediate release or enteric coated “delayed” release zinc and zinc salts such as zinc acetate, zinc sulphate and zinc monocysteine when formulated in a pill or capsule in a free or inorganic form, if taken by a person away from food, will disperse in the stomach, jejunum or upper duodenum in the water in which such pill or capsule was swallowed. Such zinc containing supplement, given away from food will induce metallothionein in the jejunum and duodenum and block the absorption and reabsorption of copper in tap water or released into the bile by the body. If such zinc moieties are given in high enough daily dose (i.e. greater than 10 mg per day) for a sufficient extended period of time (i.e. more than 3-6 months), such zinc containing supplement while protecting an individual from free copper in tap water may also create an undesirable state of copper deficiency or hypocupremia in some persons. To avoid such situation, oral high dose zinc containing multi-vitamins often contain an add back of copper (for example of 2 mg per day, such as in Preservision, iCaps and Ocuvite brand ocular vitamins). Such oral high dose zinc with added-back copper have the underdesirable effect of contributing to the serum free copper pool upon dissolution in the gastrointestinal system by bypassing normal liver metabolism, a significant portion of which enters the serum directly as free copper. Such free copper is toxic and can also cross the blood brain barrier and cause neurodegenerative and other diseases.

Accordingly, an object of the present invention is a mineral supplement that contains an immediate or delayed release zinc (so as to protect the stomach from upset due to the release of zinc in the stomach, especially away from food) combined with a copper add-back, wherein such copper add-back is not in a free or inorganic form, but instead bound with cuproproteins in a bioinorganic form (and with organic plantstuff, gum or foodstuff, or milk based proteins) so that such copper add-back is slowly digested in the gastrointestinal tract and slowly transported bound to amino acids or in a free form via the portal vein to the liver for processing and incorporation into ceruloplasmin and made available via the serum bound to ceruloplasmin. The present invention includes not just sustained release forms of copper but sustained release forms of copper bound to organic ligands that require normal digestive processes to break down and make such copper available. The intended effect of such supplement is to allow the free serum copper of an individual to be eliminated via the bile while permitting ceruloplasmin bound copper to be replenished in serum via the liver.

In a preferred embodiment, the present invention comprises an enteric coated mineral supplement providing 15-200 mg of immediate release zinc that releases immediately upon dissolution in the jejunum (such zinc preferably contained in a bilayer tablet containing an immediate release form of zinc in the outer layer with the entire tablet coated with Eudragit L-100, for example) and wherein the inner layer of such tablet contains approximately 1 to 2 mg of copper bound to milk proteins in an inner core of such bilayer tablet bound with a digestible binding agent such as guar gum, xanthan or synthetic sustained polymer such as Eudragit RS, RL, or NM).

In a preferred embodiment, an oral mineral supplement contains an immediate or sustained release zinc together with sustained release iron. In a further preferred embodiment, such sustained release iron is in a bioinorganic form bound to proteins that bind iron.

The following non-limiting examples illustrate addition and/substitution of sustained release copper, iron and/or zinc optionally including molybdenum and/or sulfur to commercially available multi-vitamin formulations:

Copper, iron and/or zinc cysteine are bound to milk proteins following the teachings of Example 11 of U.S. patent application Ser. No. 11/621,962, filed 10 Jan. 2007, and published on 6 Sep. 2007 as patent publication no. US2007/0207191 A1, and International Patent Application No. PCT/US2007/060345, filed 10 Jan. 2007, and published on 26 Jul. 2007 as patent publication no. WO 2007/084818 A2. The bound complexes are dried, and mixed with various amounts of vitamins and trace elements, and formed into a pill or tablet to form a multi-vitamin/mineral food supplement.

### EXAMPLE 1

Two mg of sustained release bound copper are added to the following minerals and vitamins in preferably the same proportion as Centrum Silver, and formulated into a pill as follows (the sustained release copper replaces the copper otherwise in Centrum Silver):

Centrum ® Silver ®
Supplement Facts

| Serving Size 1 Tablet | |
|---|---|
| Each Tablet Contains | % DV |
| Vitamin A 3500 IU (29% as Beta Carotene) | 70% |
| Vitamin C 60 mg | 100% |
| Vitamin D 400 IU | 100% |
| Vitamin E 45 IU | 150% |
| Vitamin K 10 mcg | 13% |
| Thiamin 1.5 mg | 100% |
| Riboflavin 1.7 mg | 100% |
| Niacin 20 mg | 100% |
| Vitamin B6 3 mg | 150% |
| Folic Acid 400 mcg | 100% |
| Vitamin B12 25 mcg | 417% |
| Biotin 30 mcg | 10% |
| Pantothenic Acid 10 mg | 100% |
| Calcium 200 mg | 20% |
| Phosphorus 48 mg | 5% |
| Iodine 150 mcg | 100% |
| Magnesium 100 mg | 25% |
| Zinc 15 mg | 100% |
| Selenium 20 mcg | 29% |
| Copper 2 mg | 100% |
| Manganese 2 mg | 100% |
| Chromium 150 mcg | 125% |
| Molybdenum 75 mcg | 100% |
| Chloride 72 mg | 2% |
| Potassium 80 mg | 2% |
| Boron 150 mcg | * |
| Nickel 5 mcg | * |
| Silicon 2 mg | * |
| Vanadium 10 mcg | * |
| Lutein 250 mcg | * |
| Lycopene 300 mcg | 0 |

### EXAMPLES 2-160

Formulations containing sustained release copper, iron, zinc, molybdenum and/or sulfur are combined with the commercially available multi-vitamin and food supplements set forth in the table at pages 35-54 of U.S. Provisional Patent Application Ser. No. 60/894,388, filed 12 Mar. 2007. Further, in especially preferred embodiments, immediately or substantially immediately available zinc is included in these formulations, and sustained release copper and/or sustained release iron are included as well.

The preceding specific embodiments are illustrative of the practice of the present invention. It is to be understood, however, that other formulations may be formed without departing from the spirit and scope of the invention.

Incorporated herein by reference are all patents and references mentioned herein and in patent applications mentioned herein.

All measurements disclosed herein are at standard temperature and pressure, at sea level on Earth, unless indicated otherwise. All materials used or intended to be used in a human being are biocompatible, unless indicated otherwise.

The foregoing embodiments are presented by way of example only; the scope of the present invention is to be limited only by the following claims.

1. A multi-vitamin preparation containing immediate or delayed release free zinc and delayed or sustained release protein bound copper.

2. The preparation of claim 1, wherein the protein bound copper is bound to an amino acid of a protein.

3. The preparation of claim 1, wherein the protein bound copper is covalently bound to an amino acid of a protein or a metalloprotein with sufficient binding to survive the acidic environment of the stomach.

4. A multi-vitamin preparation containing immediate or delayed release free zinc and delayed or sustained release protein bound copper and sustained released protein bound iron.

5. A multi-vitamin preparation containing delayed or sustained release protein bound copper and delayed or sustained released zinc.

6. A multi-vitamin preparation containing delayed or sustained release protein bound copper, delayed or sustained release protein bound iron, and delayed or immediate release free zinc.

7. A multi-vitamin preparation containing delayed or sustained release protein bound iron and delayed or sustained release zinc.

8. The composition of claim 1, wherein the delayed or sustained release copper, delayed or sustained release iron and/or delayed or sustained release zinc are enteric coated.

9. The composition of claim 1, wherein the delayed or sustained release copper, delayed or sustained release iron and/or delayed or sustained release zinc are bound to a pharmaceutically acceptable, stable, natural or synthetic carrier.

10. The composition according to claim 9, wherein the carrier comprises a plant fiber.

11. The composition according to claim 9, wherein the copper, iron and/or zinc are bound to low molecular weight amino acid.

12. The composition according to claim 9, wherein the copper, iron and/or zinc are dissolved in a solution of dried or evaporated milk, dried whey, dried milk lipids and dried milk proteins.

13. The composition according to claim 3, wherein the zinc comprises a zinc cysteine complex.

14. The composition according to claim 3, wherein the zinc comprises 25-75 mg delayed or sustained release zinc.

15. The composition according to claim 1, in the form of a pill or tablet.

16. The composition according to claim 1 further including delayed or sustained release molybdenum.

17. The composition according to claim 1 further including delayed or sustained release sulfur.

18. The composition according to claim 1 further including delayed or sustained release molybdenum and sulfur.

19. The invention of claim 1, comprising the components of multi-vitamins and in the amounts detailed in the Harvard Food Frequency Questionnaire without any immediate release or free or inorganic copper.

20. The invention of claim 1, in which redox active minerals including copper or iron are complexed with digestible protein, fiber, or other natural or synthetic material so as to minimize the potential bolus flux of such metals into the serum of patients.

21. The invention of claim 1, in which redox active minerals including copper or iron are in a sustained release form so as to minimize the potential bolus flux of such metals in a free form into the serum of patients.

22. A multi-vitamin preparation containing delayed or sustained release protein bound copper and zinc in a substantially immediately available form.

23. The preparation of claim 22, wherein the copper is complexed with digestible protein, fiber, or other natural or synthetic material so as to minimize the potential bolus flux of such copper into the serum of patients.

24. The preparation of claim 22, wherein the zinc is selected from the group of zinc, a zinc-cysteine complex, zinc acetate or another zinc salt.

25. The preparation of claim 24, wherein the zinc is a zinc-monocysteine complex.

26. The preparation of claim 22, wherein the zinc is in an immediately available form

27. The preparation of claim 22, wherein the zinc is bioavailable within 20 minutes after oral ingestion away from food by a human.

28. The preparation of claim 22, wherein the delayed or sustained release copper is not bioavailable until at least 60 minutes after oral ingestion away from food by a human.

29. The preparation of claim 22, wherein the zinc is bioavailable in the jejunum and duodenum of a human after oral ingestion by the human of the preparation.

30. The preparation of claim 22, wherein the delayed or sustained release copper is not bioavailable in the jejunum and duodenum of a human after oral ingestion by the human of the preparation.

31. The preparation of claim 22, wherein the delayed or sustained release copper is not bioavailable until the delayed or sustained release copper reaches the duodenum of a human after oral ingestion by the human of the preparation.

32. The preparation of claim 1, containing between about 7.5 and about 200 mg bioavailable elemental zinc and between about 0.45 and about 8 mg bioavailable elemental copper.

33. The preparation of claim 1, containing between about 16.5 and about 100 mg bioavailable elemental zinc and between about 0.725 and about 5 mg bioavailable elemental copper.

34. The preparation of claim 1, containing between about 25 and about 50 mg bioavailable elemental zinc and between about 1 and about 2 mg bioavailable elemental copper.

35. The invention of claim 1, comprising the components of any commercially available multi-vitamin supplement without any immediate release copper or free copper or inorganic copper.

36. An oral mineral preparation containing an immediate release or delayed release zinc moiety together with sustained release copper.

37. The oral mineral preparation of claim 36, wherein the mineral preparation is enteric coated.

38. The oral mineral preparation of claim 36 wherein the zinc moiety is in an inorganic form and the copper is in a bioinorganic form.

39. The oral mineral preparation of claim 36 containing 7.5 mg to 200 mg of elemental zinc.

40. The oral mineral preparation of claim 36 containing 1 mg to 8 mg of elemental copper

41. The oral mineral preparation of claim 36 further containing vitamins.

42. An oral mineral preparation containing an immediate release or delayed release zinc moiety together with sustained release iron.

43. The oral mineral preparation of claim 42, wherein the mineral preparation is enteric coated.

44. The oral mineral preparation of claim 42 wherein the zinc moiety is in an inorganic form and the iron is in a bioinorganic form.

45. The oral mineral preparation of claim 42 containing 7.5 mg to 200 mg of elemental zinc.

46. The oral mineral preparation of claim 42 containing 3 mg to 36 mg of elemental iron.

47. The oral mineral preparation of claim 42 further containing vitamins.

48. A multimineral dietary supplement composition for oral administration containing, per unit dose:
(a) an outer layer having a zinc in a coating that is a quicker release portion that is adapted to be released in the upper gastrointestinal tract, and
(b) a slow release copper core component, present in controlled release form, so adapted so as to be subsequently released in a controlled manner lower in the gastrointestinal tract.

49. A multimineral composition according to claim 48, wherein the formulation is in the form of a tablet.

50. A multimineral composition according to claim 49, wherein said tablet also contains a protective film coating surrounding said outer layer of one or both components.

51. A multimineral composition according to claim 48, wherein component (a) contains between about 7.5 and about 200 mg bioavailable zinc.

52. A multimineral composition according to claim 48 wherein component (b) contains between about 1 and about 8 mg bioavailable copper.

53. A multimineral composition according to claim 48, wherein the zinc is in the form of zinc acetate, zinc sulphate or zinc monocysteine.

54. A multimineral composition according to claim 48, wherein the copper is in the form of copper bound to protein.

55. A multimineral composition according to claim 48, further comprising bioavailable magnesium in the form of magnesium oxide, magnesium hydroxide or magnesium sulfate.

56. A multimineral composition according to claim 48, wherein the zinc and copper are in a laminated tablet.
